SEQUENCE LISTING (5 sequences)

SEQ ID NO: 1 - protein, 18 aa, Homo sapiens
PAEEDTNVYTEKHSDSLF

SEQ ID NO: 2 - protein, 18 aa, Mus musculus
PAIKSTDVYTEKHSDNLF

SEQ ID NO: 3 - protein, 137 aa, Homo sapiens
MYLDNSSIEEASGVYPIDDDDYASASGSGADEDVESPELTTSRPLPKILLTSAAPKVETTTLNIQNKIPAQTKSPEETDKEKVHLSDSERKMDPAEEDTNVYTEKHSDSLFKRTEVLAAVIAGGVIGFLFAIFLILL

SEQ ID NO: 4 - protein, 201 aa, Homo sapiens
MRRAWILLTLGLVACVSAESRAELTSDKDMYLDNSSIEEASGVYPIDDDDYASASGSGADEDVESPELTTSRPLPKILLTSAAPKVETTTLNIQNKIPAQTKSPEETDKEKVHLSDSERKMDPAEEDTNVYTEKHSDSLFKRTEVLAAVIAGGVIGFLFAIFLILLLVYRMRKKDEGSYDLGERKPSSAAYQKAPTKEFYA

SEQ ID NO: 5 - protein, 202 aa, Mus musculus
MQRAWILLTLGLMACVSAETRTELTSDKDMYLDNSSIEEASGVYPIDDDDYSSASGSGADEDIESPVLTTSQLIPRIPLTSAASPKVETMTLKTQSITPAQTESPEETDKEEVDISEAEEKLGPAIKSTDVYTEKHSDNLFKRTEVLAAVIAGGVIGFLFAIFLILLLVYRMRKKDEGSYDLGERKPSSAAYQKAPTKEFYA

(19) United States
(12) Patent Application Publication
(10) Pub. No.: US 2021/0113654 A1
(43) Pub. Date: Apr. 22, 2021

(54) Methods and Compositions to Alleviate Vascular Permeability

(71) Applicant: YALE UNIVERSITY, New Haven, CT (US)

(72) Inventors: Michael Simons, New Haven, CT (US); Federico Corti, New Haven, CT (US)

(21) Appl. No.: 17/058,348

(22) PCT Filed: May 30, 2019
(86) PCT No.: PCT/US2019/034644

§ 371 Date: Nov. 24, 2020

Related U.S. Application Data
(60) Provisional application No. 62/678,493, filed on May 31, 2018.

(51) Int. Cl. A61K 38/17; A61K 38/10; A61K 47/64; C07K 16/28; C12N 15/113; A61K 31/7105; A61K 38/46; A61K 9/00

(52) U.S. Cl. CPC A61K 38/177; A61K 38/10; A61K 47/64; C07K 16/2896; C12N 2310/14; A61K 31/7105; A61K 38/465; A61K 9/0048; C12N 15/1138

(57) ABSTRACT

In various aspects and embodiments the invention provides compositions and methods useful in the treatment of diseases related to vascular permeability. In another aspect, the invention provides a method of treating a vascular permeability related disease in a subject, the method comprising administering to the subject an effective amount of a syndecan-2 disrupting agent.

### CROSS REFERENCE TO RELATED APPLICATIONS

The present application claims priority under 35 U.S.C. § 119(e) to U.S. Provisional Patent Application No. 62/678,493 filed May 31, 2018, which is incorporated herein by reference in its entirety.

### STATEMENT REGARDING FEDERALLY SPONSORED RESEARCH OR DEVELOPMENT

This invention was made with government support under HL062289 awarded by National Institutes of Health. The government has certain rights in the invention.

### BACKGROUND OF THE INVENTION

Vascular leakage associated with inflammation and tissue injury is a factor in a wide variety of pathologies. See Lange et al., Nature Reviews Neurology, published online Jul. 1, 2016. Vascular endothelial growth factor (VEGF) plays a significant role in regulating vascular permeability with significant negative and positive effects on the health of the subject. There is a need in the art for methods and compositions for ameliorating the negative effects of VEGF without disrupting the positive effects. The present disclosure addresses this need.

### SUMMARY OF THE INVENTION

In one aspect, the invention provides a method of reducing vascular permeability in a subject, the method comprising administering to the subject an effective amount of a syndecan-2 disrupting agent.

In another aspect, the invention provides a method of treating a vascular permeability related disease in a subject, the method comprising administering to the subject an effective amount of a syndecan-2 disrupting agent.

In various embodiments, the vascular permeability related disease is selected from the group consisting of stroke, myocardial infarction, congestive heart failure, amyotropic lateral sclerosis, Alzheimer's disease, Huntington's disease, Parkinson's disease, peripheral neuropathies, traumatic brain injury, epilepsy and multiple sclerosis.

In various embodiments, the vascular permeability related disease is a retinopathy.

In various embodiments, the retinopathy is selected from the group consisting of age-related macular degeneration, diabetic retinopathy and retinopathy of prematurity.

In various embodiments, the syndecan-2 disrupting agent is a peptide having SEQ ID NO: 1.

In various embodiments, the syndecan-2 disrupting agent is a syndecan-2 extracellular domain having the amino acid sequence of SEQ ID NO: 3.

In various embodiments, either one of the peptide having the amino acid sequence of SEQ ID NO: 1 or the syndecan-2 extracellular domain having the amino acid sequence of SEQ ID NO:3 is conjugated to a heterologous peptide.

In various embodiments, the heterologous peptide is selected from the group consisting of a cell penetrating peptide, a secretion signal peptide or a stability enhancing domain.

In various embodiments, the syndecan-2 disrupting agent is an antibody, siRNA or a CRISPR system.

In various embodiments, the syndecan-2 disrupting agent is administered to the subject in a pharmaceutical composition comprising the syndecan-2 disrupting agent and at least one pharmaceutically acceptable carrier.

In various embodiments, the subject is a mammal.

In various embodiments, the subject is a human.

In various embodiments, the effective amount of a syndecan-2 disrupting agent is delivered by intraocular injection.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1G show that SDC2 controls VEGFA-induced vascular permeability in vivo. FIGS. 1A and 1B depict generation of SDC2 global knockout mice (FIG. 1A) and validation of RNA/protein loss (FIG. 1B). FIG. 1C shows that SDC2−/− mice have impaired VEGFA-induced vascular permeability as shown by reduction of Evans Blue skin leakage. FIGS. 1D and 1E show that the two main VEGFA isoforms (FIG. 1D depictss VEGF165 and FIG. 1E depicts VEGF121) show a similar reduction in vascular permeability.

FIGS. 1F and 1G show that baseline tissue permeability (FIG. 1F) and response to histamine (FIG. 1G) are normal in SDC2−/− mice indicating a specific defect in VEGFA-induced permeability.

FIGS. 2A-2D show that lack of SDC2 prevents BBB disruption and stroke damage. FIGS. 2A and 2B show that lack of SDC2 prevents blood-brain barrier disruption after stroke by Evans Blue leakage (FIG. 2A) and water content increase after Middle Cerebral Artery (MCA) permanent ligation (FIG. 2B). FIGS. 2C and 2D depict images (FIG. 2C) and a graph (FIG. 2D) showing that stroke infarct size is reduced by almost 50% in SDC2−/− compared to WT as shown by triphenyl tetrazolium chloride (TTC) live staining.

FIGS. 3A-3D show that SDC2−/− endothelial cells (ECs) show impaired activation of VEGFR2 permeability site (Y951). FIGS. 3A and 3B each depict an experiment showing that ECs that lack SDC2 have reduced VEGFR2 activation at the tyrosine site (Y951) that promotes vascular permeability. FIG. 3C shows that the reduction in Y951 activation is due to increased surface level of tyrosine phosphatase DEP1 in SDC2−/− ECs. FIG. 3D shows that DEP1 silencing in human umbilical vein endothelial cells (HUVEC) mainly leads to higher VEGFR2 Y951 activation following VEGF stimulation.

FIGS. 4A-4E show that SDC2 controls DEP1 surface level via extracellular domain protein-protein interaction. FIG. 4A depicts a co-immunoprecipitation (CO-IP) experiment showing that SDC2 forms a complex with DEP1. The association is PDZ-independent and specific to SDC2. FIG. 4B depicts a gel showing that increasing SDC2 expression level leads to reduction in DEP1 surface level. FIG. 4C shows that pre-treatment of endothelial cells with a Sdc2 peptide (which inhibits its interaction with DEP1 phosphatase) mimic loss of Sdc2 and lead to reduction in 951 activation. FIG. 4D depicts data showing that a commercial antibody (α95-105) that targets SDC2 region near the DEP1 interaction site leads to higher DEP1 surface level. FIG. 4E shows that the antibody as described in FIG. 4E inhibits in-vitro vascular permeability in response to VEGF stimulation.

FIGS. 5A-5D depict retina neovascularization. FIG. 5A depicts adeno-associated viruses (AAVs) expressing Sdc2 extracellular domain injected in subretinal space followed by laser-induced retinal-injury. FIG. 5B shows that expression is confirmed by GFP fluorescence (upper) and qPCR RNA level (lower). FIG. 5C depicts images and FIG. 5D depicts a bar graph showing that retinal neovascularization is reduced by almost by 50% in the group expressing SDC2 extracellular domain.

FIGS. 6A-6D show that SDC2 HS-chains can act as a VEGFA trap. FIG. 6A depicts a Western blot showing that SDC2 acts a VEGFR2 co-receptor via its HS chains: digestion of HS chains with K5 Lyase or Heparinase I/III prevents formation of VEGFR2/SDC2 complex. FIG. 6B is a graph showing that isolated SDC2 extracellular domain binds VEGF in a plate binding assay. FIG. 6C depicts images (left) and graphs (right) showing that endothelial SDC2 expression, but not SDC4, is required for full VEGF-induced angiogenic response in a cornea pocket assay. FIG. 6D shows an in vitro competition experiment where Sdc2 extracellular domain carrying heparan sulfate chains achieve VEGFA signaling inhibition (pVEGFR2). This effect contributes to inhibition of retina neovascularization.

FIG. 7 is a schematic depicting a summary of the properties of the SDC2 extracellular domain.

FIG. 8 is a schematic depicting a summary of the biology of DEP1 and SDC2 and the effects of pharmacological inhibition.

FIG. 9A depicts a scheme of full-length Sdc2 and position of DEP1-binding region inside the D1 domain. FIG. 9B depicts an alignment of human vs mouse Sdc2 amino acid sequences near DEP1-binding region. Three immunogenic sequences (mouse) in vicinity of this region were chosen and corresponding peptides (Ab1, Ab3, Ab5) were used for antibody generation.

FIGS. 10A-10C: the specificity of each developed anti-mouse Sdc2 antibodies was tested via direct ELISA. Plates were coated either with mouse Sdc2 or mouse Sdc4 (amount in x-axis) and antibody binding was evaluated by colorimetric reaction (O.D, optical-density in y-axis). All antibodies display specificity for Sdc2 compared to Sdc4. Ab3 appears to be the most sensitive antibody (FIG. 10B). FIG. 10A depicts the curves for Ab1. FIG. 10C depicts the curves for Ab5.

FIG. 11: Effect of Ab1 in VEGF-induced permeability was evaluated by Miles Assay. Ab1 was administrated I.V. with indicated dose (blood concentration in parenthesis) and left circulating for 2 hrs. Mice were then injected with 1% Evans Blue following by skin-permeability induction with VEGF or PBS as control.

FIG. 12A: Ab1 effect in stroke was evaluated by the MCA-occlusion ischemia-reperfusion model. MCA was occluded for 1 hr (ischemia) followed by 24 reperfusion and TTC staining (a). Antibody was injected at time of reperfusion. FIG. 12B: Decreased infarct size in both Sdc2 knock-out mice (Sdc2−/−) and Ab1-treated WT (WT+Ab1) was observed.

FIGS. 13A-13C: Cross-species reactivity of each developed anti-mouse Sdc2 antibodies of was tested via direct ELISA. Plates were coated either with mouse Sdc2 or human Sdc2 (amount in x-axis) and antibody binding was evaluated by colorimetric reaction (O.D, optical-density in y-axis). Modest cross-specie reactivity was observed only for Ab3 (FIG. 13B). FIG. 13A depicts the curves for Ab1. FIG. 13C depicts the curves for Ab5.

FIG. 14: Effect of developed anti-mouse Sdc2 antibodies on DEP1 surface level of human endothelial cells. Ab3 promotes accumulation of DEP1 at the cell surface while Ab1, Ab5, and Igg Control does not have such an effect. This appear consistent with ELISA results showing that only Ab3 has human cross-species reactivity.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

The term “CRISPR/Cas” or “clustered regularly interspaced short palindromic repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via RNA-guided DNA cleavage.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

As used herein, the term “heterologous peptide” refers to any peptide, polypeptide or protein whose sequence is selected in such a way that the product of the fusion of this sequence has a sequence different from the wild-type sequence flanking the peptide to which it is fused.

As used herein, “syndecan-2” or “SDC2” may refer to the protein having the sequence:

Human
SEQ ID NO: 4
MRRAWILLTLGLVACVSAESRAELTSDKDMYLDNSSIEE
ASGVYPIDDDDYASASGSGADEDVESPELTTSRPLPKIL
LTSAAPKVETTTLNIQNKIPAQTKSPEETDKEKVHLSDS
ERKMDPAEEDTNVYTEKHSDSLFKRTEVLAAVIAGGVIG
FLFAIFLILLLVYRMRKKDEGSYDLGERKPSSAAYQKAP
TKEFYA
Mouse
SEQ ID NO: 5
MQRAWILLTLGLMACVSAETRTELTSDKDMYLDNSSIEE
ASGVYPIDDDDYSSASGSGADEDIESPVLTTSQLIPRIP
LTSAASPKVETMTLKTQSITPAQTESPEETDKEEVDISE
AEEKLGPAIKSTDVYTEKHSDNLFKRTEVLAAVIAGGVI
GFLFAIFLILLLVYRMRKKDEGSYDLGERKPSSAAYQKA
PTKEFYA

for the human and mouse homologs, or the gene encoding this protein.

A “syndecan-2 disrupting agent” as used herein, means an agent that interferes with the action of syndecan-2, as a non-limiting example by degrading the syndecan-2 protein or interfering with its production, or by preventing the binding of syndecan-2 to a ligand, as a non-limiting example, by blocking the binding site. In various embodiments, the syndecan-2 disrupting agent prevents Dep1-syndecan-2 interaction.

As used herein, the term “syndecan-2 extracellular domain” refers to a peptide having the sequence of the extracellular domain of syndecan-2 and including its associated heparan sulfate chains, either isolated or linked to a heterologous peptide. The amino acid sequence of the extracellular domain of human syndecan-2 is SEQ ID NO:3:

```
                10 20 30 40
MYLDNSSIEE ASGVYPIDDD DYASASGSGA DEDVESPELT
                50 60 70 80
TSRPLPKILL TSAAPKVETT TLNIQNKIPA QTKSPEETDK
                90 100 110 120
EKVHLSDSER KMDPAEEDTN VYTEKHSDSL FKRTEVLAAV
              130
IAGGVIGFLF AIFLILL
```

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

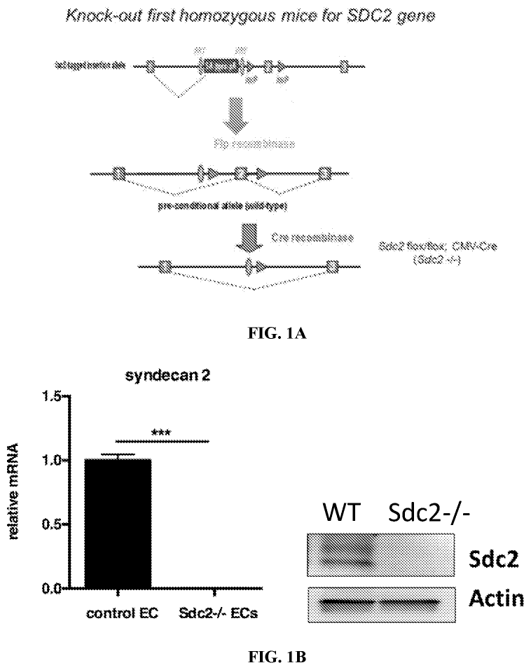

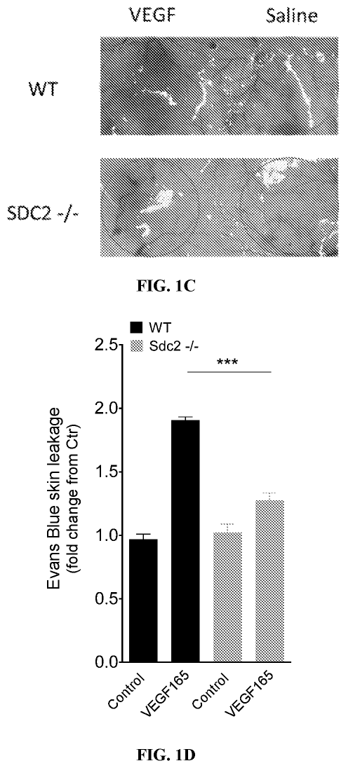

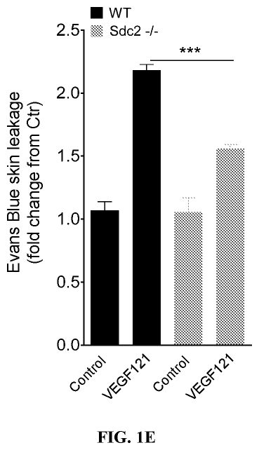

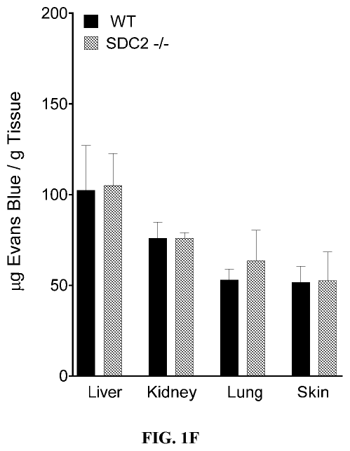

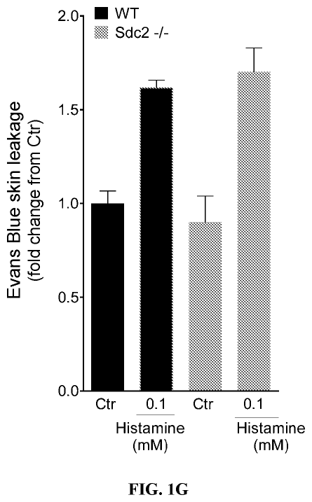

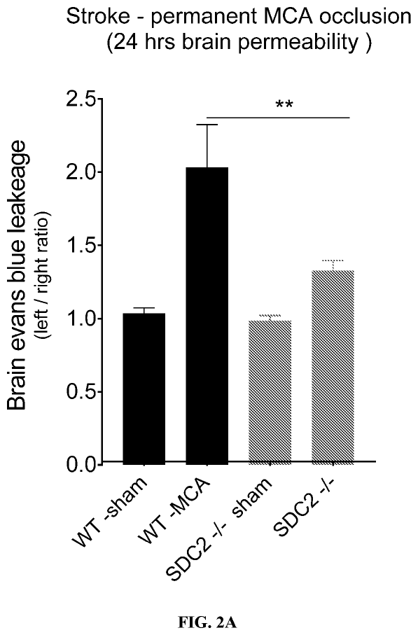

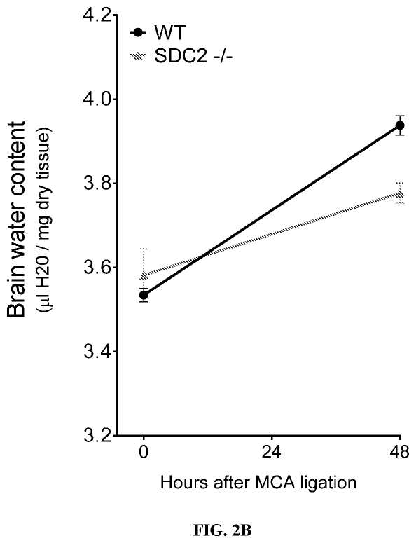

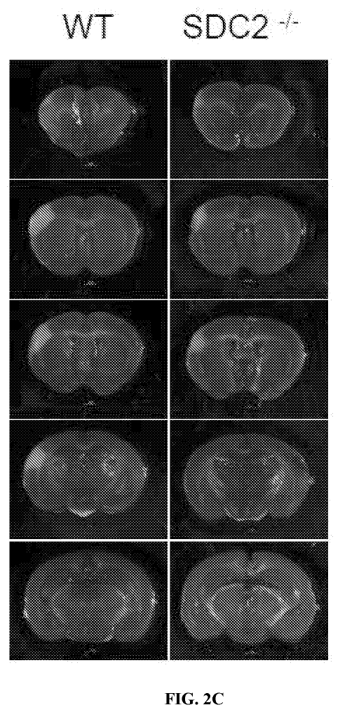

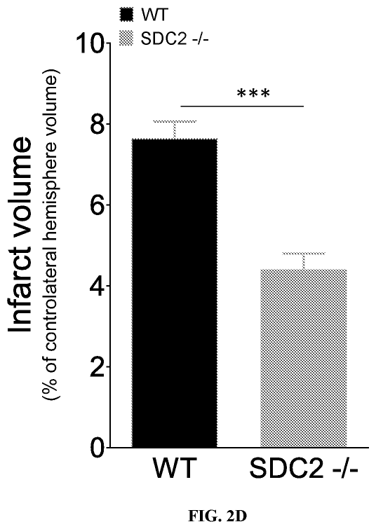

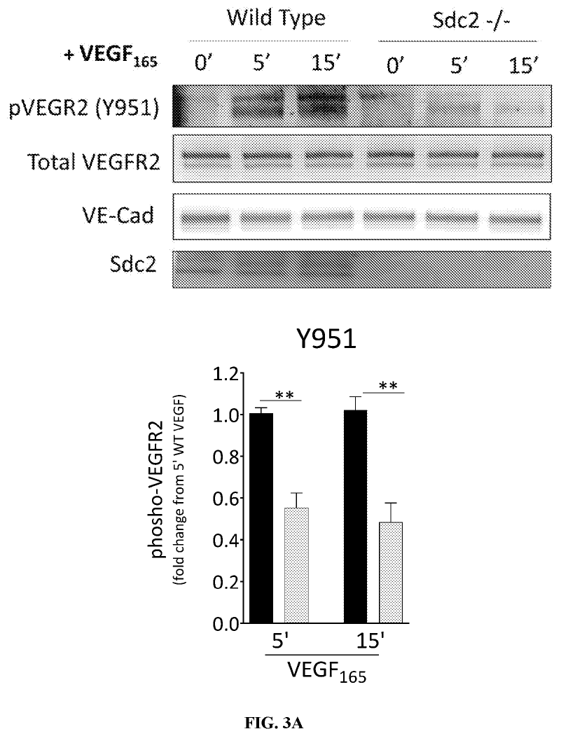

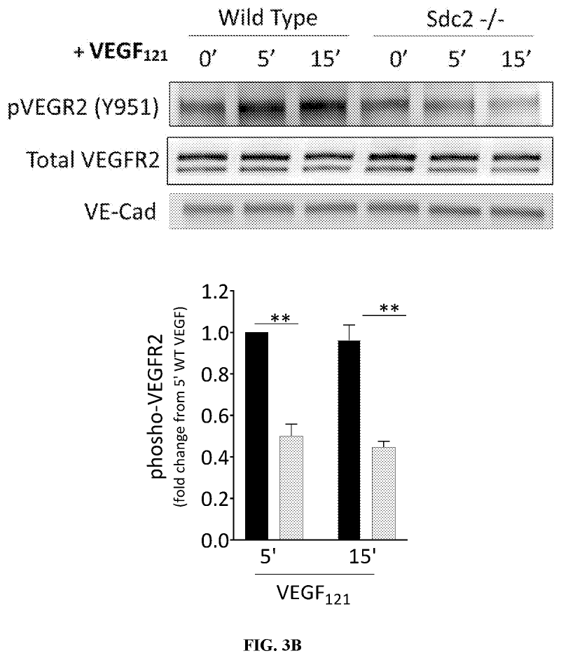

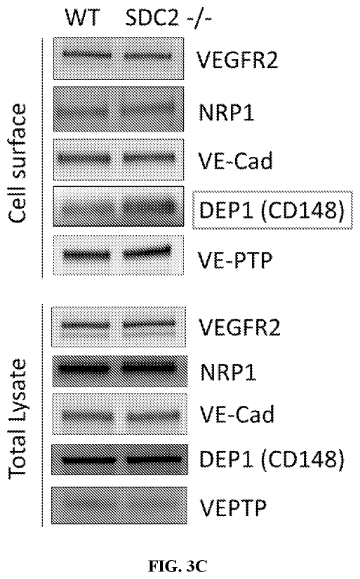

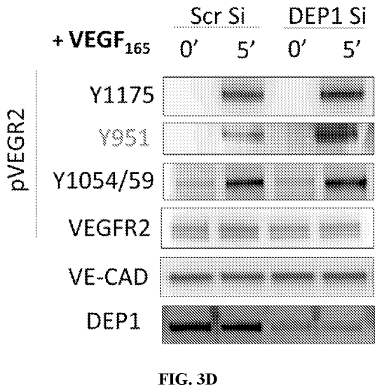

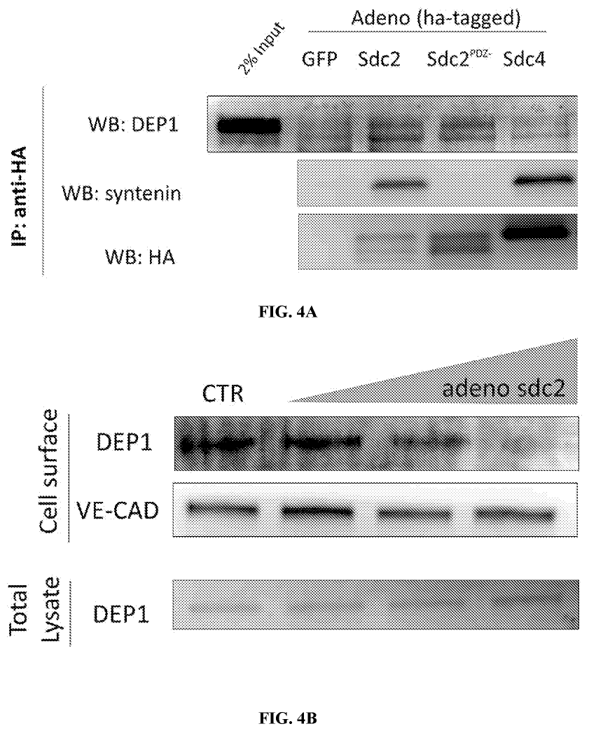

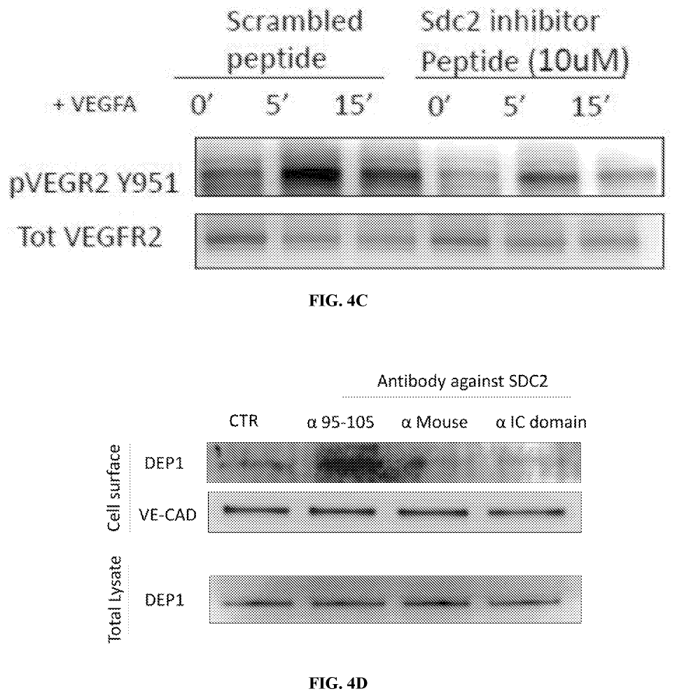

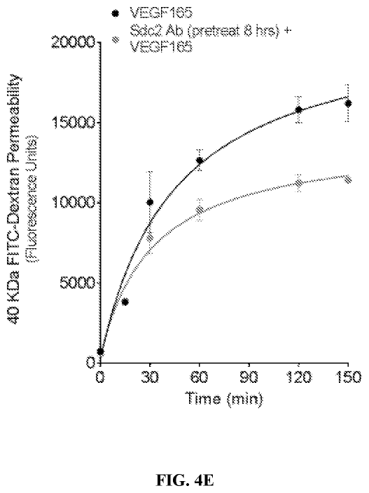

The invention is based in part on the discovery that SDC-2-Dep1 interaction controls VEGFA-induced vascular permeability, as shown in FIGS. 1A-1G, using a knock-out mouse model. Without wishing to be limited by theory, the mechanism appears to be that the extracellular domain of Sdc2 interacts with a protein tyrosine phosphatase (PTP) Dep1; Dep1 specifically dephosphorylates Y951 on VEGFR2. This site mediates permeability response induced by VEGFA. In the absence of Sdc2, Dep1 levels on the plasma membrane are increased leading to Y951 dephosphorylation and inhibition of vascular leak while leaving other VEGFR2 signaling pathways intact. This could be extremely useful for preventing disruption of the blood-brain barrier in the wake of various diseases where damage is associated with vascular permeability, and thereby preventing damage to subjects. FIGS. 2A-2D validate this approach and demonstrate substantial reduction in damage in the knock-out relative to wild-type.

Accordingly, in one aspect, the invention provides a method of reducing vascular permeability in a subject, the method comprising administering to the subject an effective amount of a syndecan-2 disrupting agent. In another aspect, the invention provides a method of treating a vascular permeability related disease in a subject, the method comprising administering to the subject an effective amount of a syndecan-2 disrupting agent.

The vascular permeability related disease may be any disease in which vascular permeability contributes to the pathology of the disease. In various embodiments the vascular permeability related disease is selected from the group consisting of stroke, myocardial infarction, congestive heart failure, amyotropic lateral sclerosis, Alzheimer's disease, Huntington's disease, Parkinson's disease, peripheral neuropathies, traumatic brain injury, epilepsy and multiple sclerosis.

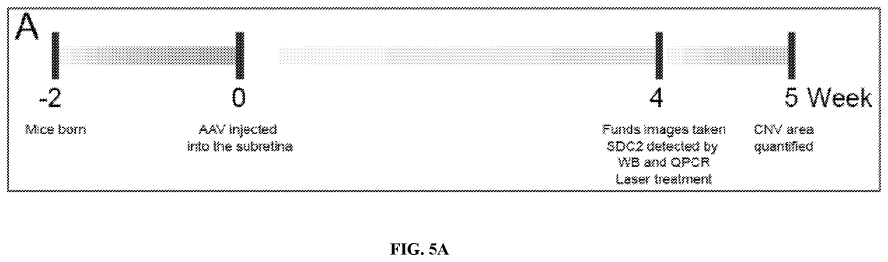

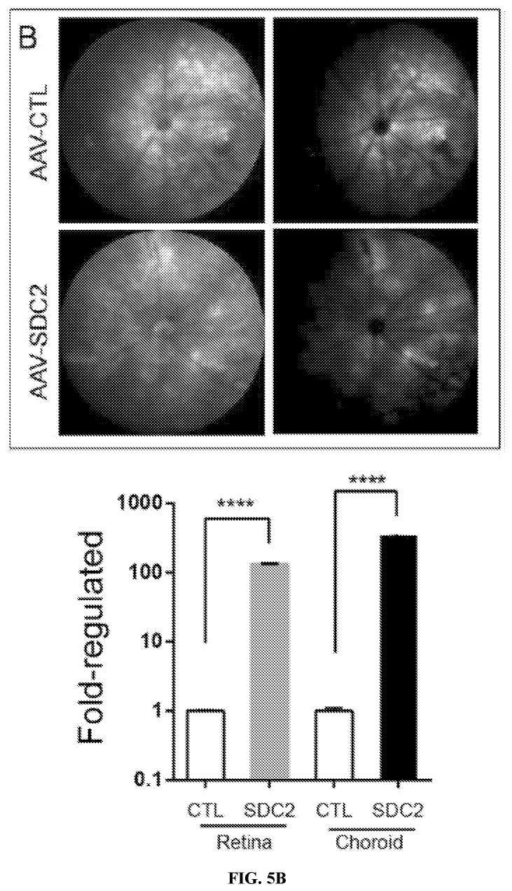

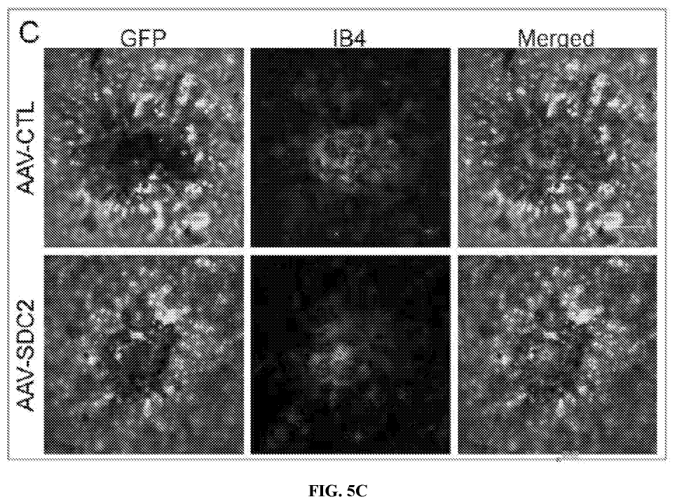

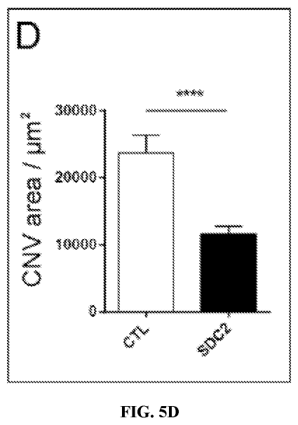

Consistent with the finding that interaction with SDC2 influences VEGFA, FIGS. 5A-D show a substantial reduction in neovascularization following retinal-injury with expression of secreted SDC2 extracellular domain delivered by expression from an AAV vector. Accordingly, in another aspect the invention provides a method of treating a retinopathy in a subject, the method comprising administering to the subject an effective amount of a syndecan-2 disrupting agent. In various embodiments, the retinopathy is a wet, neovascular stage retinopathy. In various embodiments the retinopathy is age-related macular degeneration, diabetic retinopathy, or retinopathy of prematurity.

A skilled person will understand that there are various methods of disrupting SDC2's role in the mechanism of the relevant disease processes. One approach is the use of a soluble SDC2 extracellular domain. In various embodiments, the syndecan-2 disrupting agent is a peptide having SEQ ID NO: 1 PAEEDTNVYTEKHSDSLF, corresponding to SDC2 extracellular domain amino acids 123-140. The analogous peptide in mouse is SEQ ID NO: 2 PAIKSTDVYTEKHSDNLF corresponding to mouse syndecan-2 region 124-141.

In various embodiments, the syndecan-2 disrupting agent further comprises a heterologous peptide. In various embodiments, the heterologous peptide may be a cell penetrating peptide, by way of non-limiting example a transactivator of transcription (TAT) peptide, a secretion signal peptide, by way of non-limiting example a preprotyrypsin signal sequence or a stability enhancing peptide. The stability enhancing peptide may be any peptide that extends the syndecan-2 disrupting agent's half-life in vivo relative to the syndecan-2 disrupting agent alone.

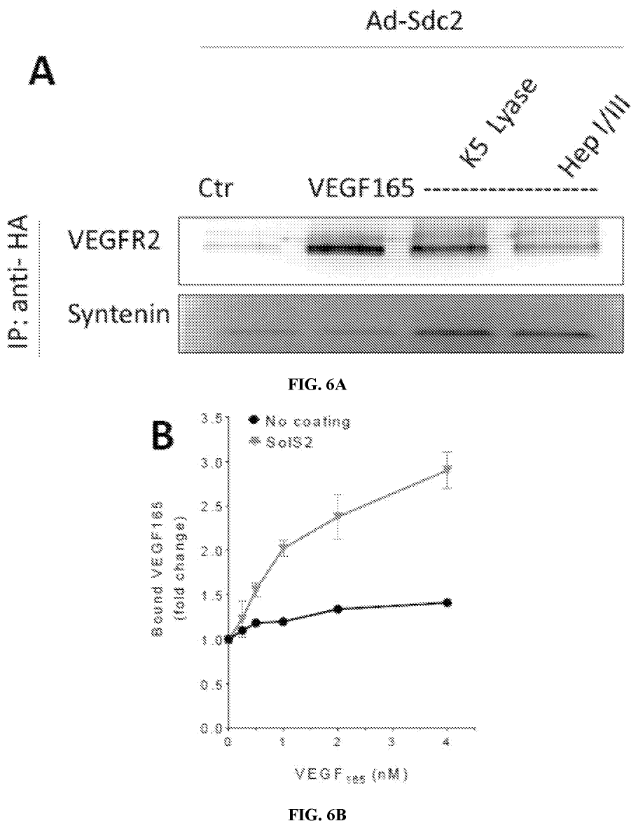

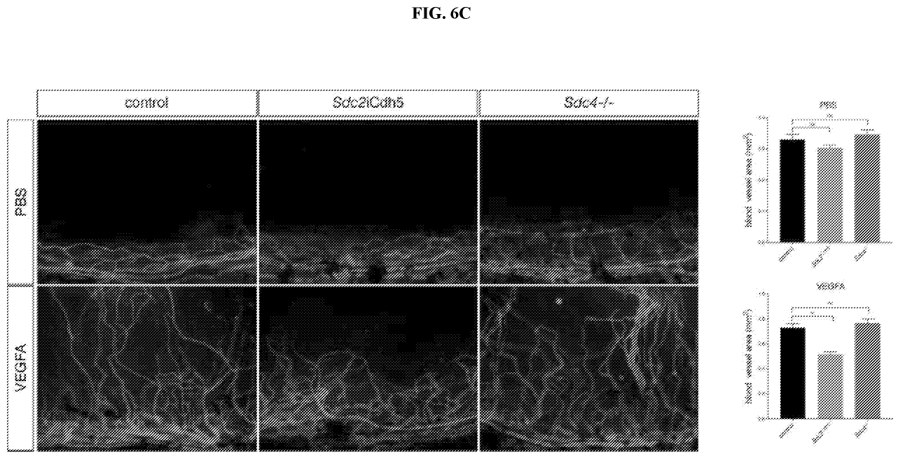

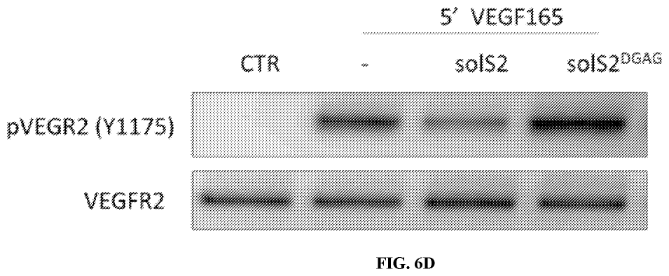

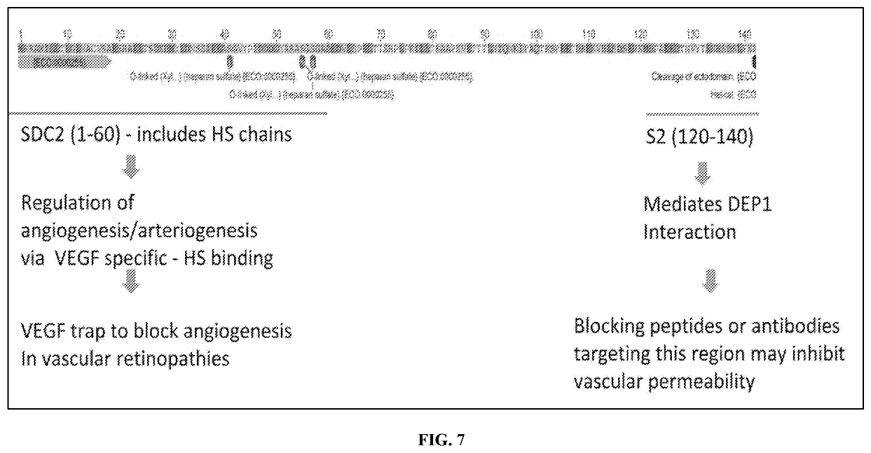

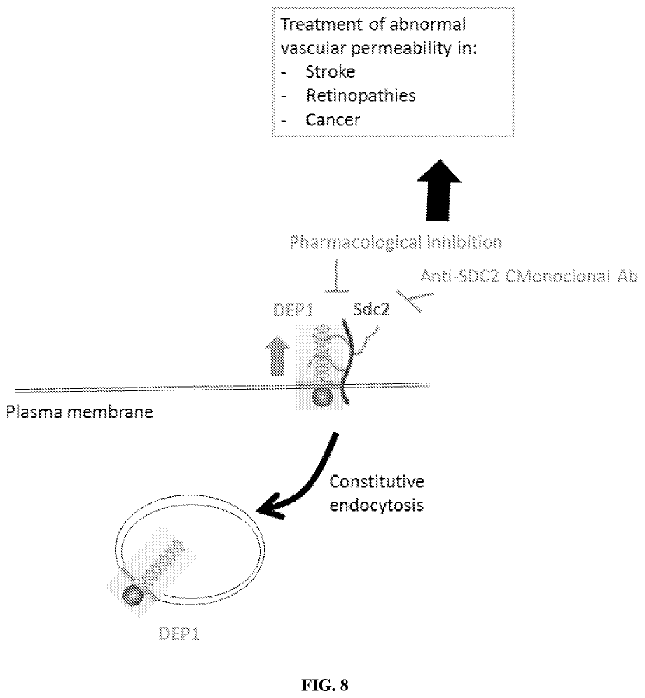

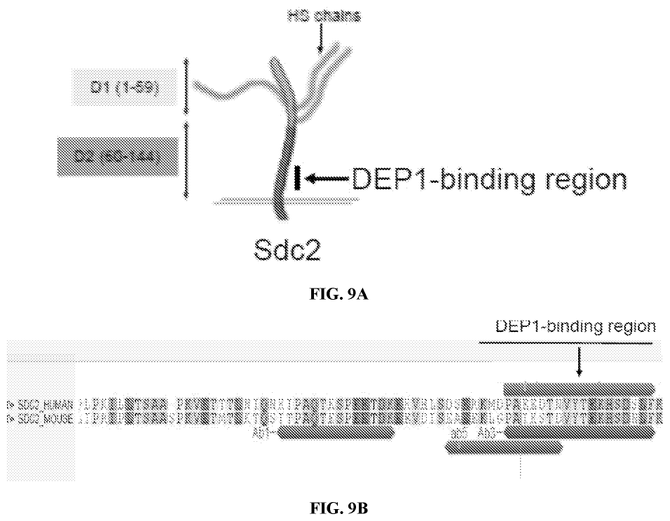

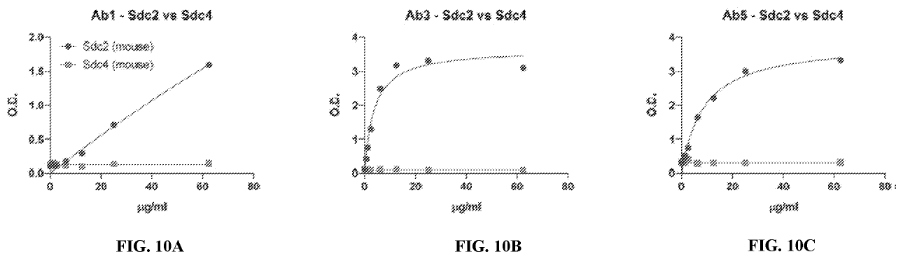

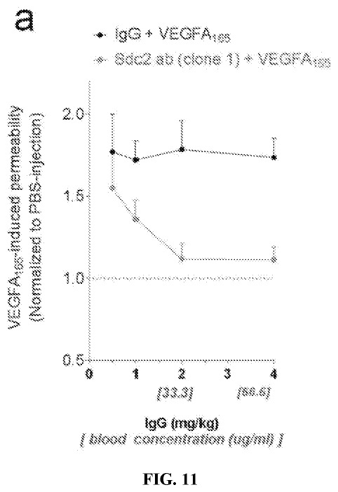

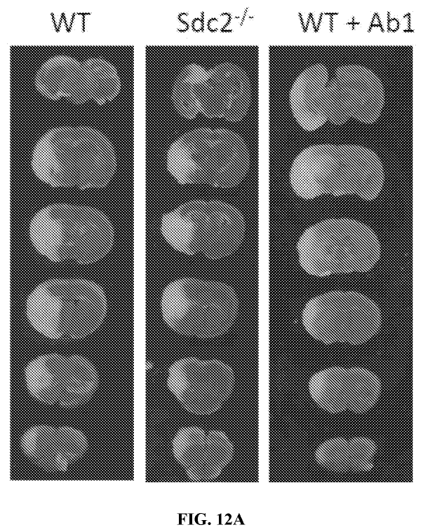

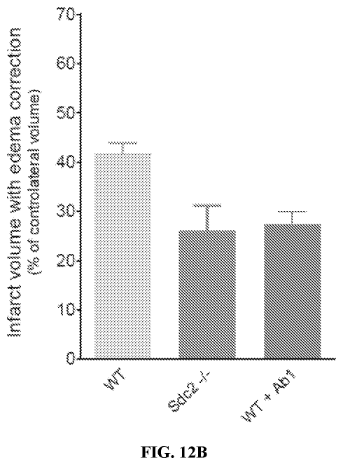

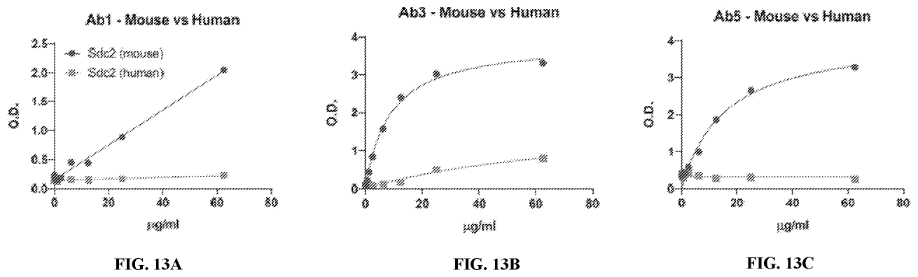

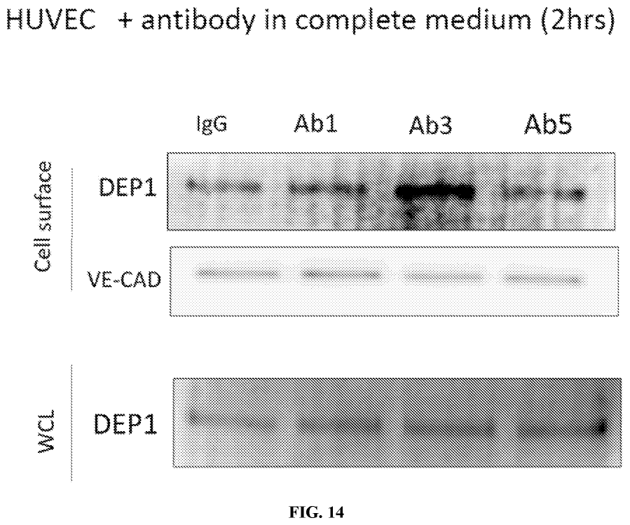

FIG. 6D shows that SDC2 heparan sulfate chains can function as VEGFA trap and reduce VEGFR2 activation. Accordingly, in various embodiments, the syndecan-2 disrupting agent may be the extracellular domain which include at least amino acids 1-60 and its associated heparan sulfate chains. In various embodiments, the syndecan-2 disrupting agent may be chemically isolated heparan sulfate chains from syndecan-2.

Agents that reduce the level of SDC2 may also be syndecan-2 disrupting agents. RNA interference may be used to suppress the SDC2 gene and reduce levels of SDC2. In various embodiments, the syndecan-2 disrupting agent is a small interfering RNA targeting SDC2 mRNA. Alternatively, gene editing may be used to suppress syndecan-2 or disrupt the DEP1 binding site. In various embodiments, the syndecan-2 disrupting agent is a CRISPR system.

Another strategy is to use an antibody targeting the SDC2-Dep1 binding site. Polyclonal antibodies targeting this site are available commercially (ThermoFisher Scientific, Catalog number: 7101835, target region: 95-105 in human syndecan-2). Accordingly, in various embodiments the syndecan-2 disrupting agent is an antibody.

In various embodiments, the syndecan-2 disrupting agent is administered to the subject in a pharmaceutical composition comprising the syndecan-2 disrupting agent and at least one pharmaceutically acceptable carrier. In various embodiments, the subject is a mammal. In various embodiments, the subject is a human. In various embodiments, the effective amount of a syndecan-2 disrupting agent is delivered by intraocular injection. Appropriate pharmaceutical dosage forms are discussed elsewhere herein.

### CRISPR/Cas9

The CRISPR-Cas9 system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 2931 cells), primly cells, and CAR I cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identities and cleaves target sequences. Cas9 is comprised of six domains: REC I, REC II, Bridge Helix, PAM interacting, HNH, and RuvC. The Reel domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HMI nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US20140068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to stem cells and immune cells (Addgene, Cambridge, Mass., Minis Bio LLC, Madison, Wis.).

The guide RNA is specific for a genomic region of interest and targets that region for Cas endonuclease-induced double strand breaks. The target sequence of the guide RNA sequence may be within a loci of a gene or within a non-coding region of the genome. In certain embodiments, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In other embodiments, the CRISPR/Cas system is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

### Administration/Dosage/Formulations

The regimen of administration may affect what constitutes an effective amount. The therapeutic formulations may be administered to the subject either prior to or after the onset of a disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a patient.

The carrier may be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The present invention also includes a multi-layer tablet comprising a layer providing for the delayed release of one or more compounds of the invention, and a further layer providing for the immediate release of a medication for treatment of certain diseases or disorders. Using a wax/pH-sensitive polymer mix, a gastric insoluble composition may be obtained in which the active ingredient is entrapped, ensuring its delayed release.

Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Dosing

The therapeutically effective amount or dose of a compound of the present invention depends on the age, sex and weight of the patient, the current medical condition of the patient and the progression of a disease in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of reducing vascular permeability in a subject, the method comprising administering to the subject an effective amount of a syndecan-2 disrupting agent.

2. A method of treating a vascular permeability related disease in a subject, the method comprising administering to the subject an effective amount of a syndecan-2 disrupting agent.

3. The method of claim 2, wherein the vascular permeability related disease is selected from the group consisting of stroke, myocardial infarction, congestive heart failure, amyotropic lateral sclerosis, Alzheimer's disease, Huntington's disease, Parkinson's disease, peripheral neuropathies, traumatic brain injury, epilepsy and multiple sclerosis.

4. The method of claim 2, wherein the vascular permeability related disease is a retinopathy.

5. The method of claim 4, wherein the retinopathy is selected from the group consisting of age-related macular degeneration, diabetic retinopathy and retinopathy of prematurity.

6. The method of claim 1, wherein the syndecan-2 disrupting agent is a peptide having the amino acid sequence of SEQ ID NO: 1.

7. The method of claim 1, wherein the syndecan-2 disrupting agent is a syndecan-2 extracellular domain having the amino acid sequence of SEQ ID NO:3.

8. The method of claim 6, wherein the peptide having the amino acid sequence of SEQ ID NO: 1 is conjugated to a heterologous peptide.

9. The method of claim 8, wherein the heterologous peptide is selected from the group consisting of a cell penetrating peptide, a secretion signal peptide or a stability enhancing domain.

10. The method of claim 1, wherein the syndecan-2 disrupting agent is an antibody, siRNA or a CRISPR system.

11. The method of claim 1, wherein the syndecan-2 disrupting agent is administered to the subject in a pharmaceutical composition comprising the syndecan-2 disrupting agent and at least one pharmaceutically acceptable carrier.

12. The method of claim 1, wherein the subject is a mammal.

13. The method of claim 1, wherein the subject is a human.

14. The method of claim 4, wherein the effective amount of a syndecan-2 disrupting agent is delivered by intraocular injection.

15. The method of claim 7, wherein the syndecan-2 extracellular domain having the amino acid sequence of SEQ ID NO: 3 is conjugated to a heterologous peptide.
